(19) United States
(12) Patent Application Publication
(10) Pub. No.: US 2024/0174944 A1
(43) Pub. Date: May 30, 2024

(54) NOVEL CYCLIC ACETAL AND CARBONYL-CONTAINING FRAGRANCE MOLECULES

(71) Applicants: Sean William Raspet, Detroit, MI (US); Shengping Zheng, New York, NY (US); Sihan Li, New York, NY (US)

(72) Inventors: Sean William Raspet, Detroit, MI (US); Shengping Zheng, New York, NY (US); Sihan Li, New York, NY (US)

(73) Assignee: STRUCTURE HOLDINGS LLC, Grosse Pointe Farms, MI (US)

(21) Appl. No.: 16/895,442

(22) Filed: Jun. 8, 2020

(51) Int. Cl. C11B 9/00; C07D 317/26; C07D 317/46; C07D 319/06; C07D 321/08

(52) U.S. Cl. CPC C11B 9/008; C07D 317/26; C07D 317/46; C07D 319/06; C07D 321/08; C11B 9/0076; C11B 9/0084

(57) ABSTRACT

The present disclosure provides for novel fragrance molecules, namely a range of related fragrance molecules all containing a cyclic acetal or dioxepane sub-structure in combination with a carbonyl sub-structure and a phenyl sub-structure.

Specifically, several compounds of the formula:

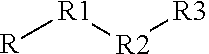

Wherein:
R=is or contains a phenyl group
R1=is or contains a methyl, ethyl, propyl, or butyl group, including branched groups or groups with additional methyl, ethyl or propyl sub-structures
R2=is or contains a cyclic acetal group or dioxepane
R3=is or contains a carbonyl group, including a ketone, methanal, ethanal, propanal, or acetyl group

### FIELD OF THE EMBODIMENTS

The present disclosure provides for novel fragrance molecules, namely a range of related fragrance molecules containing a cyclic acetal sub-structure in combination with a carbonyl sub structure.

### BACKGROUND OF THE EMBODIMENTS

In the fragrance, flavor and scented consumer products industries, there is an ongoing need for novel fragrance ingredients in order to impart new and interesting fragrance qualities to products. These novel fragrance ingredients, in particular, novel molecular compounds, can allow products to differentiate from competing products and to create, new and memorable sensory experiences for consumers. In this document, it is understood that the term fragrance also includes the related applications of flavor and other products related to olfactory and gustatory experience.

The sensory space of possible fragrance is vast and relatively unexplored. While the entirety of all possible colors in human vision are the result of the activity of three types of color receptor in the human eye, the space of scent is exponentially vaster, with an estimated ˜400 types of functioning human olfactory receptors. There are yet many undiscovered “colors” in the fragrance space.

An effective way to explore the space of fragrance is by producing novel molecular structures with novel scent properties. These allow for the production of new and useful scent-related products. Several examples of novel fragrance molecules and a method for their manufacture are disclosed herein.

Our research has discovered a molecular structure template or rule that corresponds to a range of powerful new floral odorant molecules. These compounds contain 1) phenyl group is present in a molecular structure that is then bound to 2) a cyclic acetal structure (for example: a dioxane or dioxolane) or dioxepane structure, either directly or via a chain or one or more carbon atoms, and 3) a carbonyl structure is present on or in close proximity to the cyclic acetal's ring structure. These molecules tend to have a powerful, diffusive and highly desirable floral, sweet character that is useful in fragrance and related applications.

### SUMMARY OF THE EMBODIMENTS

Several compounds of the formula:

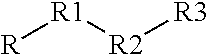

Wherein:
R=is or contains a phenyl group
R1=is or contains a methyl, ethyl, propyl, or butyl group, including branched groups or groups with additional methyl, ethyl or propyl sub-structures
R2=is or contains a cyclic acetal group or dioxepane
R3=is or contains a carbonyl group, including a ketone, methanal, ethanal, propanal, or acetyl group

And wherein the compound is novel, having not been described in prior art relating to fragrance applications.

### DESCRIPTION OF THE PREFERRED EMBODIMENTS

In the case of the chemical synthesis reactions described below it is understood that the invention may be synthesized via other reactions. The reactions given herein are provided as examples and should not limit the scope of the invention.

The embodiments detailed below are provided as examples and should not limit the scope of the invention.

### Example 1

The following example is intended to be representative of an embodiment of the present invention. A molecule of the formula:

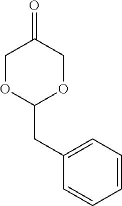 [SMILES: O=C1COC(Cc2ccccc2)OC1]

### (“2-benzyl-1,3-dioxan-5-one”)

In one embodiment, the compound above can be produced via an oxidation of 2-benzyl-1,3-dioxan-5-ol.

2-benzyl-1,3-dioxan-5-ol itself can be produced via a cyclic acetal formation between glycerol and the aldehyde phenylacetaldehyde. In this document it is understood that the term acetal is used as a general term to designate both acetals and ketals.

One of the products from the above reaction is the cyclic acetal, alcohol-containing compound: 2-benzyl-1,3-dioxan-5-ol.

This compound is then oxidized to form the embodiment 2-benzyl-1,3-dioxan-5-one.

The reaction proceeds as in the scheme below.

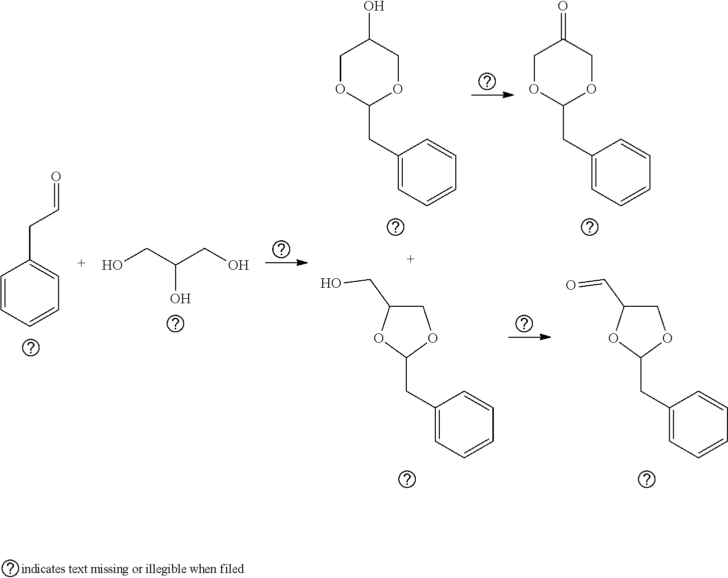 [SMILES: O=CCc1ccccc1 ; OCC(O)CO ; OCC1COC(Cc2ccccc2)O1 ; OC1COC(Cc2ccccc2)OC1 ; O=C1COC(Cc2ccccc2)OC1 ; O=CC1COC(Cc2ccccc2)O1]

2-benzyl-1,3-dioxan-5-one has a very pleasant, characteristic rose-floral aspect. It has been described by some or our reviewers as identical to the smell of a petal of a rose flower with a highly realistic aspect. Others have said it perfectly captures the scent of fresh spring air as mimosa flowers begin to bloom. It is surprisingly much stronger than its starting material and additionally has a much more full and natural-seeming aspect.

The compound is very powerful and diffusive which can offer the advantage of a lower use percentage and thus a lower cost-in-use in formulations. In addition to its use in formulations it can potentially be used as a stand-alone note or single-molecule fragrance.

In other compositions it may provide a base for or aspects of spring air, honeysuckle and other fresh, floral-sweet notes.

In one embodiment, the acetal formation reaction proceeds via toluene reflux. glycerol and phenylacetaldehyde are added in a molar ratio of 2:1. p-toluenesulfonic acid is added at a molar ratio of 0.2 mole as a catalyst. The components are dissolved in toluene or a suitable solvent and the mixture is heated to approximately 110 C. using a Dean-Stark apparatus to remove water resulting from the reaction. The reaction proceeds from 4 hours to overnight. The reaction mixture is then purified and separated to isolate 2-benzyl-1,3-dioxan-5-ol.

The reaction mixture may contain an alternative glyceryl acetal isomer: (2-benzyl-1,3-dioxolan-4-yl)methanol. This compound is also a useful product in its own right and is further detailed herein as a starting material for another embodiment of the invention.

In this embodiment, after 2-benzyl-1,3-dioxan-5-ol is isolated it is oxidized using Dess-Martin Periodinane as an oxidizing reagent. The ratio of Dess-Martin Periodinane to 2-benzyl-1,3-dioxan-5-ol used is 1.5:1. The resulting ketone, 2-benzyl-1,3-dioxan-5-one, is then isolated.

Alternatively, in this embodiment, the commercially available product 2-benzyl-1,3-dioxan-ol or the product “Acetal CD” can be used as a starting material. “Acetal CD” is a mixture of 2-benzyl-1,3-dioxan-5-ol and 2-benzyl-1,3-dioxolan-4-yl)methanol.

In one synthesis method “Acetal CD” (5 g, 25.7 mmol) is dissolved in 100m1 dichloromethane then added Dess-Martin periodinane (17g, 40 mmol). The mixture solution is then placed under reflux conditions for 12 hours, then poured the solution into 200 mL of ice-cold water and swirled for 10 minutes. After white precipitate forms, the clear solution is collected by vacuum filtration. The clear solution is extracted with 2*20 ml dichloromethane. The organic phase is then washed with brine, dried by Na2SO4, and evaporated under reduced pressure. The residue is purified by flash chromatography (Hexane/EtOAc=20:1) to give 2-benzyl-1,3-dioxan-5-one.

### Alternative Embodiments

### Example 2

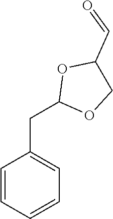 [SMILES: O=CC1COC(Cc2ccccc2)O1]

### (“2-benzyl-1,3-dioxolane-4-carbaldehyde”)

The above compound can be prepared as a side product of the synthesis scheme (shown above) for 2-benzyl-1,3-dioxan-5-one. To date it has not been directly isolated but in combination with 2-benzyl-1,3-dioxan-5-one, it appears to act as a modifier and also provides a floral aspect. We are reducing to practice further isolation and evaluation.

It may serve as a useful product in combination 2-benzyl-1,3-dioxan-5-one, which would allow for streamlining the synthesis steps involved in isolating each compound individually. Potentially it may serve as a useful, desirable product in its own right.

### Assorted Additional Examples Structure Images:

The embodiments detailed below are meant to illustrate the range of molecules of which the invention consists and should not be considered exhaustive.

### Example 3

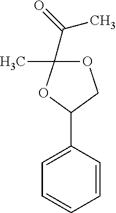

### Example 4

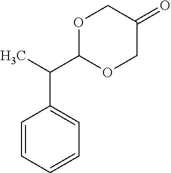

### Example 5

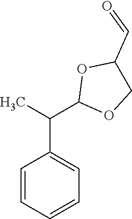

### Example 6

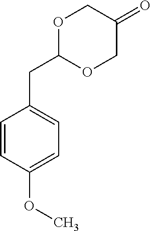 [SMILES: COc1ccc(CC2OCC(=O)CO2)cc1]

### Example 7

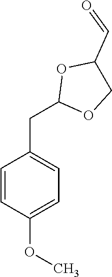 [SMILES: COc1ccc(CC2OCC(C=O)O2)cc1]

### Example 8

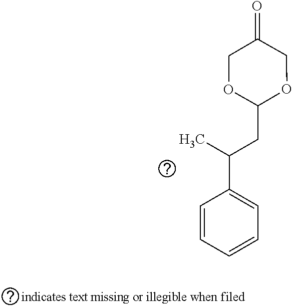 [SMILES: CC(CC1OCC(=O)CO1)c1ccccc1]

### Example 9

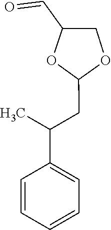 [SMILES: CC(CC1OCC(C=O)O1)c1ccccc1]

### Example 10

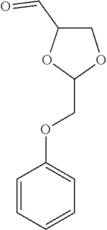 [SMILES: O=CC1COC(COc2ccccc2)O1]

### Example 11

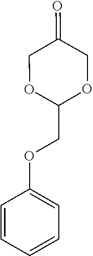 [SMILES: O=C1COC(COc2ccccc2)OC1]

### Example 12

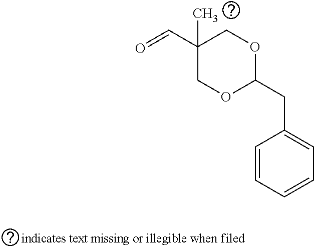 [SMILES: CC1(C=O)COC(Cc2ccccc2)OC1]

### Example 13

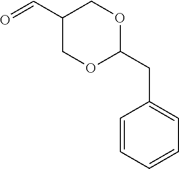 [SMILES: O=CC1COC(Cc2ccccc2)OC1]

### Example 14

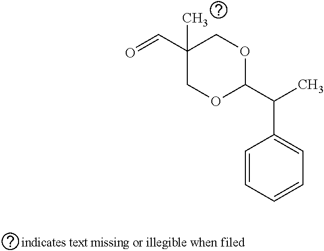 [SMILES: CC(c1ccccc1)C1OCC(C)(C=O)CO1]

### Example 15

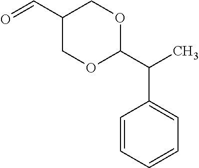 [SMILES: CC(c1ccccc1)C1OCC(C=O)CO1]

### Example 16

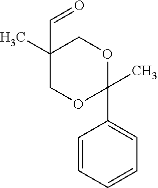

### Example 17

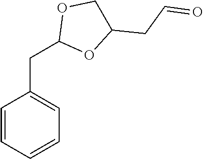 [SMILES: O=CCC1COC(Cc2ccccc2)O1]

### Example 18

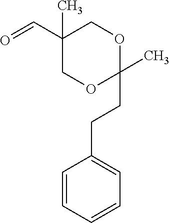 [SMILES: CC1(C=O)COC(C)(CCc2ccccc2)OC1]

### Example 19

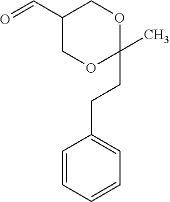 [SMILES: CC1(CCc2ccccc2)OCC(C=O)CO1]

### Example 20

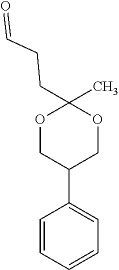 [SMILES: CC1(CCC=O)OCC(c2ccccc2)CO1]

### Example 21

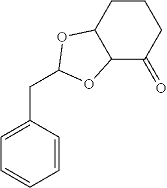 [SMILES: O=C1CCCC2OC(Cc3ccccc3)OC12]

### Example 22

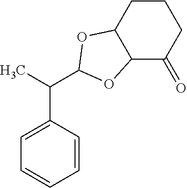 [SMILES: CC(c1ccccc1)C1OC2CCCC(=O)C2O1]

### Example 23

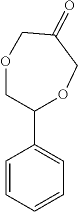 [SMILES: O=C1COCC(c2ccccc2)OC1]

### Example 24

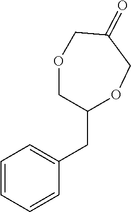 [SMILES: O=C1COCC(Cc2ccccc2)OC1]

### Example 25

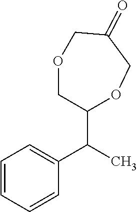 [SMILES: CC(c1ccccc1)C1COCC(=O)CO1]

### Example 26

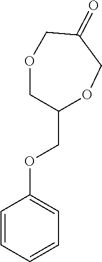 [SMILES: O=C1COCC(COc2ccccc2)OC1]

### Example 27

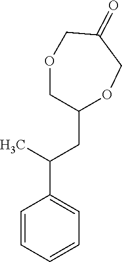 [SMILES: CC(CC1COCC(=O)CO1)c1ccccc1]

### Example 28

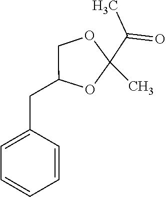 [SMILES: CC(=O)C1(C)OCC(Cc2ccccc2)O1]

### Example 29

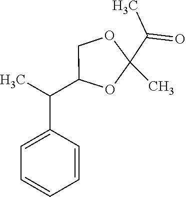 [SMILES: CC(=O)C1(C)OCC(C(C)c2ccccc2)O1]

### Example 30

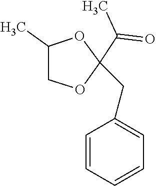 [SMILES: CC(=O)C1(Cc2ccccc2)OCC(C)O1]

### Example 31

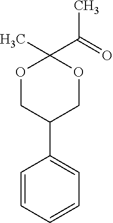 [SMILES: CC(=O)C1(C)OCC(c2ccccc2)CO1]

What we claim is:

1. A compound of formula: 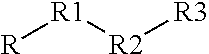
Wherein:
R=is or contains a phenyl group
R1=is or contains a methyl, ethyl, propyl, or butyl group, including branched groups or groups with additional methyl, ethyl or propyl sub-structures
R2=is or contains a cyclic acetal group or dioxepane group
R3=is or contains a carbonyl group, including a ketone, methanal, ethanal, propanal, or acetyl group
And wherein the compound is novel, having not been described in prior art relating to fragrance applications.

2. A fragrance or flavor product containing a molecule from claim 1

3. A product containing a compound from claim 1 wherein the molecule imparts or modifies the flavor or fragrance of the product

4. Stereoisomers, enantiomers and stereochemically defined isomers of compounds from claim 1

5. A fragrance or flavor product containing a molecule from claim 4

6. A product containing a compound from claim 4 wherein the molecule imparts or modifies the flavor or fragrance of the product
